(19) United States
(12) Patent Application Publication
(10) Pub. No.: US 2014/0037719 A1
(43) Pub. Date: Feb. 6, 2014

(54) STABILIZED PHARMACEUTICAL FORMULATIONS OF A POTENT HCV INHIBITOR

(71) Applicant: Robert Majeska, New York, NY (US)

(72) Inventors: Mathias BRAUN, Sohren (DE); Carl Alan BUSACCA, Poughkeepsie, NY (US); Feng-Jing CHEN, Irvine, CA (US); Edwin Louis GUMP, New Fairfield, CT (US); Jenness B. MAJESKA, (US); Scott PENNINO, New York, NY (US); Fenghe QIU, New Milford, CT (US); Maria Fernanda VILLAGRA, Buenos Aires (AR)

(73) Assignee: Boehringer Ingelheim International Gmbh, Ingelheim am Rhein (DE)

(21) Appl. No.: 13/738,057

(22) Filed: Jan. 10, 2013

Related U.S. Application Data
(60) Provisional application No. 61/586,087, filed on Jan. 12, 2012.

(51) Int. Cl. A61K 31/4709

(52) U.S. Cl. CPC A61K 31/4709
USPC 424/451; 514/312; 206/204

(57) ABSTRACT

Described are various methods for stabilizing pharmaceutical formulations of a specific Hepatitis C Viral (HCV) inhibitor against the formation of a particular genotoxic degradation product. Such methods include temperature control, moisture control, excipient control, capsule shell control, basification and a reconstitution approach.

### TECHNICAL FIELD OF THE INVENTION

The present application is directed to various methods for stabilizing pharmaceutical formulations of a specific Hepatitis C Viral (HCV) inhibitor against the formation of a particular genotoxic degradation product.

### BACKGROUND OF THE INVENTION

The following Compound (1):

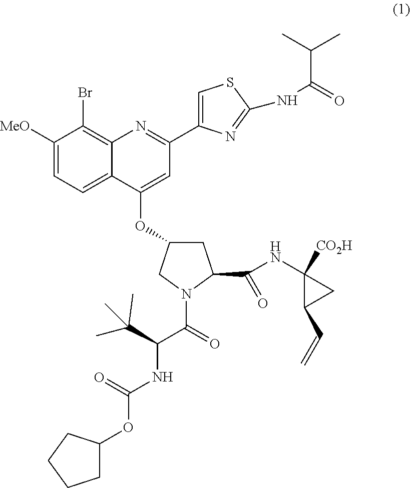 [SMILES: C=C[C@@H]1C[C@]1(NC(=O)[C@@H]1C[C@@H](Oc2cc(-c3csc(NC(=O)C(C)C)n3)nc3c(Br)c(OC)ccc23)CN1C(=O)[C@@H](NC(=O)OC1CCCC1)C(C)(C)C)C(=O)O]

having the chemical name: 1-{[4-[8-Bromo-2-(2-isopropylcarbamoyl-thiazol-4-yl)-7-methoxy-quinolin-4-yloxy]-1-(R)-(2-cyclopentyloxycarbonyl amino-3,3-(S)-dimethyl-butyryl)-pyrrolidine-(S)-2-carbonyl]-amino}-2-(S)-vinyl-cyclopropane-(R)-carboxylic acid, is known as a selective and potent inhibitor of the HCV NS3 serine protease and useful in the treatment of HCV infection. Compound (1) falls within the scope of the acyclic peptide series of HCV inhibitors disclosed in U.S. Pat. Nos. 6,323,180, 7,514,557 and 7,585,845. Compound (1) is disclosed specifically as Compound #1055 in U.S. Pat. No. 7,585,845, and as Compound #1008 in U.S. Pat. No. 7,514,557. Compound (1), and pharmaceutical formulations thereof, can be prepared according to the general procedures found in the above-cited references, all of which are herein incorporated by reference in their entirety. Preferred forms of Compound (1) include the pharmaceutically acceptable salts thereof and crystalline forms thereof, and in particular the crystalline sodium salt form as described in U.S. Patent Application Publication No. 2010/0093792, also incorporated herein by reference. The sodium salt form of Compound (1) (referred to herein at “Compound (1) NA”) in currently in clinical trials for the treatment of HCV infection.

One type of pharmaceutical formulation that has been developed for formulating Compound (1) NA is a Self-Emulsifying Drug Delivery (SEDDs) formulation in the form of a liquid-filled soft-gel capsule packaged in induction sealed HDPE bottles. Examples of this type of formulation can be found in U.S. Patent Application Publication No. US 2011/0160149. It has been discovered that upon storage of this formulation a potentially genotoxic degradation product, referred to herein as “Compound X”, is formed from the parent drug molecule via the amide hydrolysis reaction shown below in Scheme I. Another type of pharmaceutical formulation that has been developed is an oral solution formulation designed for pediatric use, and it has been confirmed that this formulation is also prone to the formation of the Compound X degradation product during storage. Examples of this type of formulation can be found in WO 2010/059667. While amide hydrolysis is a known mechanism of degradation, it was not intuitive or expected that this specific degradation product would form in these formulations and that this specific degradation product would also be an Ames positive compound and genotoxic. In fact, Compound X was not predicted to be genotoxic based on standard in-silico prediction software analysis. This unexpected discovery constitutes one aspect of the present invention.

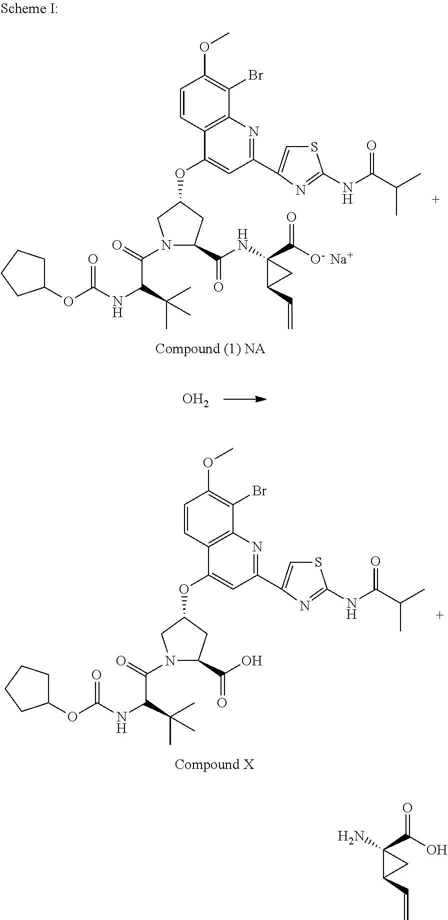

Compound X may also be depicted by the following chemical structure showing the stereochemistry at the two chiral centers in this molecule:

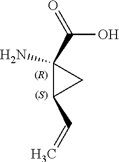

Due to the high potential toxicity of the Compound X, the increase in this impurity over the product shelf life duration was deemed unacceptable from a regulatory perspective and thus there was an urgent need to solve this problem. For example, the EMEA (European Medicines Agency) Guideline on the Limits of Genotoxic Impurities (28 Jun. 2006) specifies a maximum intake value of 1.5 μg/day of a genotoxic impurity as being associated with an acceptable risk (1 in 100,000 increased cancer risk) for most marketed pharmaceuticals based on a lifetime exposure duration. For short duration treatment regimens higher levels of genotoxic impurities may be acceptable based on application of Haber's rule (fundamental concept in toxicology) to extrapolate acceptable limits for daily intake for shorter treatment durations (Felter et al, Critical Reviews in Toxicology, 2011) without changing the associated level of cancer risk. For example, in its subsequent guidance document issued on 26 Jun. 2008, the EMEA's CHMP Safety Working Party indicated that the acceptable limits for daily intake of genotoxic impurities during clinical trials (1 in 1 million increased cancer risk plus an additional dose rate correction factor of 2) are 5, 10, 20, and 60 μg/day for a duration of exposure of 6-12 months, 3-6 months, 1-3 months, and less than 1 month, respectively. Since the treatment regimen with Compound (1) NA may be as short as 12 weeks (˜3 months) or 24 weeks (˜6 months), maximum allowable intake values for Compound X may be as high as 20 μg/day (3 month regimen) or 10 μg/day (6 month regimen) when applying a 1 in 1 million increased cancer risk and a dose rate correction factor of 2. Taking into consideration the benefit of an approved marketed product, the maximum allowable intake values for Compound X may be as high as the calculated acceptable limit of 400 μg/day (3 month regimen) or 200 μg/day (6 month regimen) when applying a 1 in 100,000 increased cancer risk level. Thus, one goal of the present invention was to develop techniques to ensure that the maximum intake value of this degradation product would be maintained below these regulatory limits.

Prior to the discovery that Compound X was an Ames positive degradation product, the stability of Compound (1) NA drug products was controlled by standard product packaging (HDPE bottle with induction seal) and room temperature storage. Such conditions were considered sufficient to allow for the desired commercial product shelf life. As noted above, current regulatory requirements for controlling potentially genotoxic impurities limit such impurities to levels much lower than standard impurities. The discovery that Compound X was Ames positive and genotoxic required the development of further controls to insure the lowest possible levels of Compound X in the drug product for patient safety and to meet requirements of regulatory authorities.

### BRIEF SUMMARY OF THE INVENTION

The present application is directed to various methods for controlling the level of the degradation product Compound X in liquid pharmaceutical compositions comprising Compound (1), or a pharmaceutically acceptable salt thereof, and to the resulting stabilized pharmaceutical compositions.

In a general embodiment, the present invention is directed to a method for controlling the level of degradation product Compound X in a liquid pharmaceutical composition comprising a Compound (1), or a pharmaceutically acceptable salt thereof, and at least one pharmaceutically acceptable excipient, said method comprising one or more of the following:
(a) drying said composition such that it has a water content of less than about 3.0% w/w and storing the composition under conditions sufficient to maintain a water content of less than about 3.0% w/w;
(b) storing said composition at a temperature of between about 2 and 8 degrees Celsius;
(c) adding a basifier to said composition to achieve an internal apparent pH of greater than about 7; or
(d) if the liquid pharmaceutical composition is to contain water as an excipient material, preparing a first formulation pre-mixture comprising only Compound (1), or pharmaceutically acceptable salt thereof, and non-aqueous based excipients and a second formulation pre-mixture comprising water as an excipient, and then mixing the first and second formulation pre-mixtures to prepare the final formulation just prior to patient use.

Additional embodiments are directed to liquid pharmaceutical composition comprising a Compound (1), or a pharmaceutically acceptable salt thereof, and at least one pharmaceutically acceptable excipient, wherein the amount of degradation product Compound X in the composition is below a level of about 400 μg, or below a level of about 200 μg, or below a level of about 60 μg, or below a level of about 20 μg, when the composition contains a full daily dose of Compound (1) or pharmaceutically acceptable salt thereof in either single or multiple dosage units. And in a more specific embodiment, the composition has one or more of the following properties:
(a) a water content of less than about 3.0% w/w;
(b) an internal temperature of between about 2 and 8 degrees Celsius; or
(c) an internal apparent pH of greater than about 7.

Additional embodiments are directed to the above methods and compositions wherein the total resulting amount of degradation product in the composition is below a level of about 1.5 μg when the composition contains a full daily dose of Compound (1), or pharmaceutically acceptable salt thereof, in either single or multiple dosage units.

### BRIEF DESCRIPTION OF THE DRAWINGS

FIG. 1 depicts the Compound X formation and stability over a 24 month period under different temperature conditions for a batch of 120 mg capsules of Compound (1) NA.

FIG. 2 depicts the Compound X formation and stability over a 24 month period at room temperature for 120 mg capsules of Compound (1) NA having different levels of fill water content.

FIG. 3 shows the combined effect of storage temperature and fill water content for 120 mg capsules of Compound (1) NA on Compound X formation and stability over a 12 month period.

FIG. 4A is a graphic representation of a blister packaging system incorporating a desiccant in the product packaging, and the affect on water transmission.

FIG. 4B shows a more detailed depiction of a capsule in an exemplary polymer blister packaging system and the water transmission in such system

FIG. 5 depicts the changes in relative humidity over time within the pouch and within the polymer blister cavity and the changes in moisture content within the capsule fill formulation for a packaging system comprising an aluminum pouch containing a conditioned desiccant and capsules enclosed in a polymer blister.

FIG. 6 shows Compound X stability over a 24 month period under refrigerated conditions (4-5° C.) for 120 mg capsules of Compound (1) NA having different levels of fill water content,

FIG. 7 shows three different oral solution formulations of Compound (1) NA designed for employing three different degradation product control methods of the present invention.

### DETAILED DESCRIPTION OF THE INVENTION

### Definitions

Terms not specifically defined herein should be given the meanings that would be given to them by one of skill in the art in light of the disclosure and the context. As used throughout the present application, however, unless specified to the contrary, the following terms have the meaning indicated:

The term “about” means within 5%, and more preferably within 1% of a given value or range. For example, “about 3.7%” means from 3.5 to 3.9%, preferably from 3.66 to 3.74%. When the term “about” is associated with a range of values, e.g., “about X % to Y %”, the term “about” is intended to modify both the lower (X) and upper (Y) values of the recited range. For example, “about 20% to 40%” is equivalent to “about 20% to about 40%”.

The term “pharmaceutically acceptable salt” means a salt of a Compound of formula (1) which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use.

The term includes pharmaceutically-acceptable acid addition salts and pharmaceutically-acceptable base addition salts. Lists of suitable salts are found in, e.g., S. M. Birge et al., J. Pharm. Sci., 1977, 66, pp. 1-19.

The term “pharmaceutically-acceptable acid addition salt” means those salts which retain the biological effectiveness and properties of the free bases and which are not biologically or otherwise undesirable, formed with inorganic acids such as hydrochloric acid, hydrobromic acid, sulfuric acid, sulfamic acid, nitric acid, phosphoric acid, and the like, and organic acids such as acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethane-sulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid, and the like.

The term “pharmaceutically-acceptable base addition salt” means those salts which retain the biological effectiveness and properties of the free acids and which are not biologically or otherwise undesirable, formed with inorganic bases such as ammonia or hydroxide, carbonate, or bicarbonate of ammonium or a metal cation such as sodium, potassium, lithium, calcium, magnesium, iron, zinc, copper, manganese, aluminum, and the like. Particularly preferred are the ammonium, potassium, sodium, calcium, and magnesium salts. Salts derived from pharmaceutically-acceptable organic nontoxic bases include salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins, and the like. Particularly preferred organic to nontoxic bases are isopropylamine, diethylamine, ethanolamine, trimethylamine, dicyclohexylamine, choline, and caffeine.

### Formation of the Compound X Degradation Product

As noted previously, the basis for the present invention was the discovery that a specific degradation product, Compound X, is formed upon storage of a liquid formulation containing Compound (1) NA and that this specific degradation product is Ames positive and genotoxic. This was unknown prior to the present invention. As a direct result of this discovery, it became apparent that additional control methods would be necessary to control the level of Compound X formation in the drug product in order to meet regulatory requirements.

It is known that the rates of many chemical reactions increase with temperature and moisture. For this reason, several commercial SEDDs capsule formulations e.g. tipranavir soft gel capsules, are stored under refrigeration or protected from moisture using induction sealed packaging in order to provide for increased stability and/or shelf life. For the Compound (1) NA SEDDs drug product, following the discovery that Compound X is formed and is genotoxic, the kinetics of the formation of this degradation product were studied and were found to be a function of temperature. Investigations into the mechanism of formation suggest that Compound X is formed via acid catalyzed amide hydrolysis (see Scheme I above) and so increased levels of moisture would also be expected to drive the formation of Compound X. This has been confirmed in studies of Compound (1) NA capsules manufactured with various levels of fill formulation water content. All product stability and experimental studies conducted to date have demonstrated that Compound X levels increased with higher temperatures and are significantly higher with moisture ingress. Thus, temperature and moisture control methods constitute aspects of the present invention. Additional control methods that have been discovered are discussed in detail below.

### Compound X Degradation Product Control Methods

The various control methods that have been developed to address the Compound X degradation product issue, include the following methods, each of which is discussed in more detail below:
1. Temperature Control
2. Moisture Control
3. Excipient Control
4. Capsule Shell Control
5. Basification
6. Reconstitution Approach

### 1. Temperature Control

The initial hydrolysis formation reaction that produces Compound X from Compound (1) NA has been demonstrated to be temperature dependent. As such, refrigeration can be used to reduce the rate of Compound X formation in liquid formulations by directly slowing the rate of the hydrolysis reaction. A suitable preferred temperature range for refrigeration of such liquid formulation is between about 2 and 8 degrees Celsius and as such constitutes a preferred embodiment of the invention. Under refrigerated conditions the rate of Compound X formation comes into balance with the rate of Compound X degradation (via reaction with fatty acid based excipients or other degradation mechanisms, as discussed in detail below) within the formulation insuring a low and controlled Compound X level in the drug product. However, even formulations that do not possess formulation excipients that react with Compound X in a sacrificial manner may still benefit from reduced Compound X levels as a result of refrigeration.

With respect to temperature control, a refrigerated supply chain is recommended for the product throughout it's shelf life and at least until the product is in the patient's hands. The product should be stored in refrigerated warehouse facilities with temperature condition monitoring to insure that product is maintained at proper temperature. During transportation, the product should be transported under refrigerated conditions. As best practice, temperature monitoring devices (e.g. TempTale® from Sensitech Inc.) should be included with shipments to provide assurance that temperature conditions during shipment do not deviate beyond known safe product temperature excursion ranges. Thus, one additional embodiment of the invention is directed to a liquid pharmaceutical composition wherein the packaged dosage units of the composition are stored together with a temperature monitoring device to measure and record the environmental temperature during storage or shipment. A temperature monitoring device is typically attached to, or otherwise included with, a larger shipment quantity, e.g. a pallet, of the packaged dosage units. All such possible temperature monitoring devices and attachments thereof to pharmaceutical drug product that are conventional in the industry are embraced by the present invention.

Temperature control can be used independently to control the formation of degradation product or, more preferably, together with one or more of the other control methods as described herein.

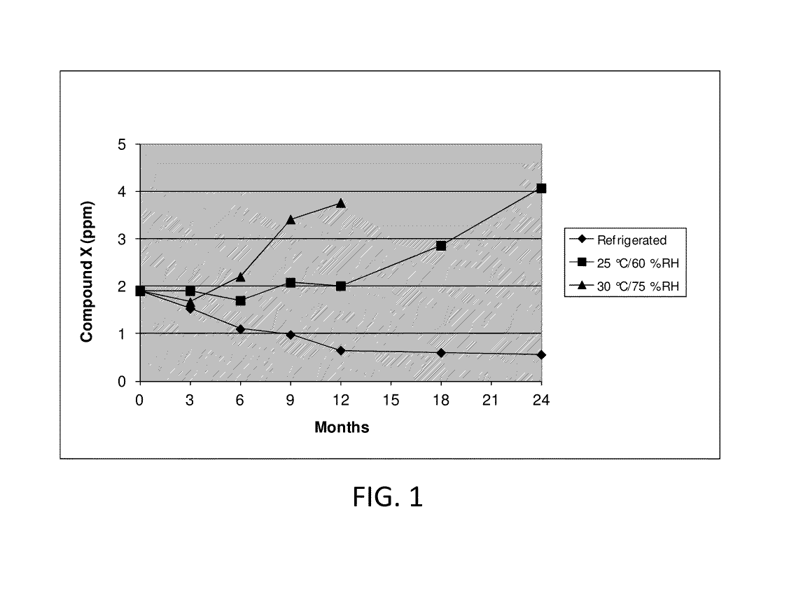

For example, the combination of refrigeration, moisture control (control drying during manufacture and moisture resistant packaging), and the presence of sacrificial fatty acid-based excipients (excipient control) provides for very effective control of Compound X. Under such conditions, Compound X levels have been demonstrated to maintain a “steady state” level of approximately 0.5-1 ppm in drug product capsules which is well below the requirements of regulatory authorities. Because the level of Compound X is at steady state, the product shelf life can be extended well beyond that possible at room temperature while insuring patient safety. To date, two years of stability (shelf life) for the product has been demonstrated on several representative batches. Compound X results over the course of these stability studies show no increasing trend suggesting that considerable further extension in product shelf life is possible. See FIG. 1, which depicts the Compound X formation and stability over a 24 month period under different environmental conditions for a batch of 120 mg capsules (where “Refrigerated”=4-5° C.). The temperature of the refrigerated environment was set to 4-5° C. The results clearly demonstrate effective degradation product control under refrigerated conditions as compared to non-refrigerated environments. In fact, the results demonstrate that the degradation product level decreases under refrigerated storage conditions and is maintained at this lower level.

Achieving a low steady state level of Compound X in Compound (1) NA capsules throughout shelf life by this invention, allows for greater opportunity for room temperature use and storage of the product with the patient for a limited period of time during patient use of the product. This accommodation for greater flexibility in patient handling of the product provides greater ease of patient use and potentially greater patient dosing compliance. It thus provides a significant advantage versus requiring the patient to store the product in a refrigerator. Preliminary studies under conditions of simulated patient use have shown that Compound X does not exceed commercial regulatory limits even after 60 days of storage at 25° C./70% RH or 30° C./75% RH. See Table 1 below:

TABLE 1
Compound X in Compound (1) NA Capsules (120 mg) During Simulated Patient Use:

| | Timepoint | | | | | | |
|---|---|---|---|---|---|---|---|
| | Time | | | | | | |
| | 0 | 30 Days | | | 60 Days | | |
| | Condition | | | | | | |
| | | | 25° C./ | 30° C./ | | 25° C./ | 30° C./ |
| | NA | 5° C. | 60% RH | 75% RH | 5° C. | 60% RH | 75% RH |
| Compound X (ppm) | 1.3 | 0.8 | 0.8 | 1.0 | 1.3 | 1.4 | 2.4 |

Implementation of temperature control methods does not negatively impact other quality aspects of the product. As drug solubility in the formulation increases upon lowering temperature, this product can be stored under refrigeration without impacting assay.

Furthermore, the capsules maintain their physical properties such as hardness and show an improved overall product degradation profile.

### 2. Moisture Control

Compound X is formed via hydrolysis and, as such, the concentration of water present in the solution/fill formulation has a direct impact on the rate of Compound X formation. As the rate of hydrolytic Compound X formation from Compound (1) NA is dependent on the presence of water to drive the reaction, ultra dry formulations can minimize Compound X formation. This has been demonstrated, for example, by the development of the Compound (1) NA oral solution drug product formulation that consists of two solutions designed to be mixed at time of patient use: Compound (1) NA dissolved in a dry solvent (e.g. PEG400 and propylene glycol) and an excipient vehicle containing water. This is the so-called “Reconstitution Approach” method described hereinbelow as an alternative technique to minimize the effect of water in the formulation.

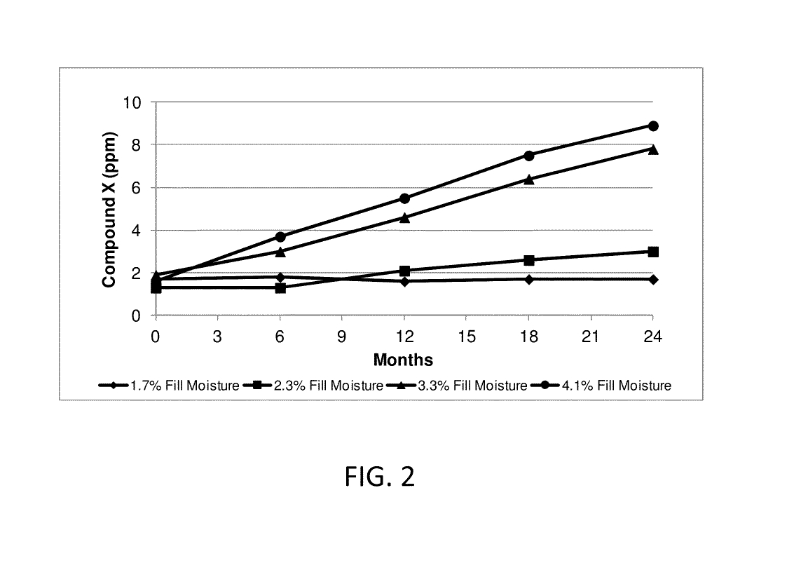

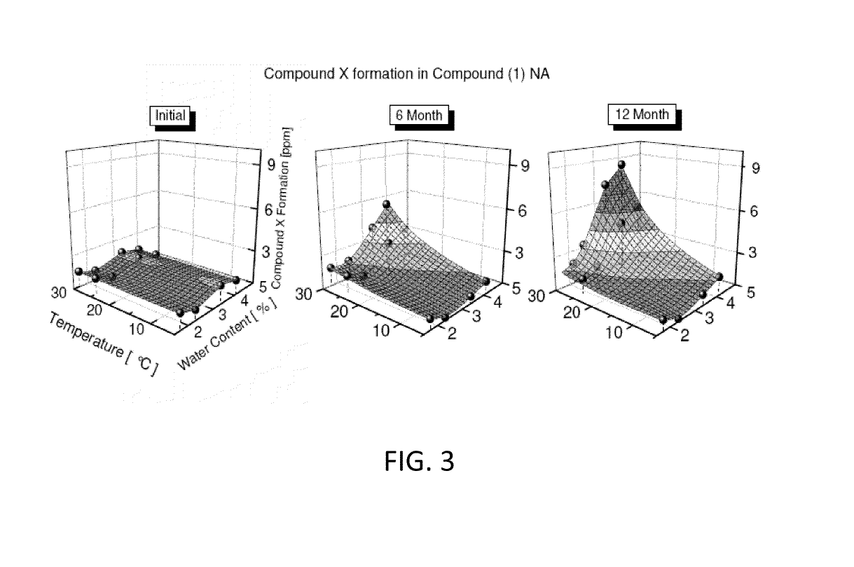

Investigation of Compound (1) NA capsules manufactured with different levels for water content has demonstrated that at very low levels of water, Compound X growth can be effectively controlled even at room temperature storage. See FIG. 2, which depicts the Compound X formation and stability over a 24 month period at room temperature storage for 120 mg capsules having different levels of water content in the fill formulation. When such data are also evaluated with the consideration of temperature, the results indicate that for capsules with the lowest water content, Compound X growth shows relatively little temperature dependency (see FIG. 3), and can therefore be maintained below regulatory limits even at room temperature storage. As such, low water content formulations might be suitably stable to Compound X formation and not require refrigeration or other temperature controls.

Unfortunately, at such low water levels, current soft gelatin capsule shell material becomes highly brittle and lacks sufficient robustness to avoid cracking when packaged and shipped in bottles. It may be possible to develop alternative capsule formulations that are sufficiently elastic and robust even when filled with low water content formulations to produce Compound (1) NA capsules with commercially acceptable physical robustness to be packaged and distributed in bottles.

In a preferred embodiment, SEDDSs capsules are dried to less than about 3.0% water content during manufacturing and then stored under conditions suitable to maintain such water content level. At this water content, refrigeration effectively controls Compound X formation in the formulation while insuring that the capsule shell possesses sufficient elasticity to be robust for product packaging and distribution. Additional embodiments include drying the capsules to less than about 2.5% water content, or less than about 2.0% water content, and then storing the capsules under conditions suitable to maintain such water content level. Drying methods that may be used include any of the conventional drying methods known in the art, including but not limited to adsorption drying or condensation heat drying. For capsules, a typical drying method is in drying tunnels at 20-25° C./10-15% RH.

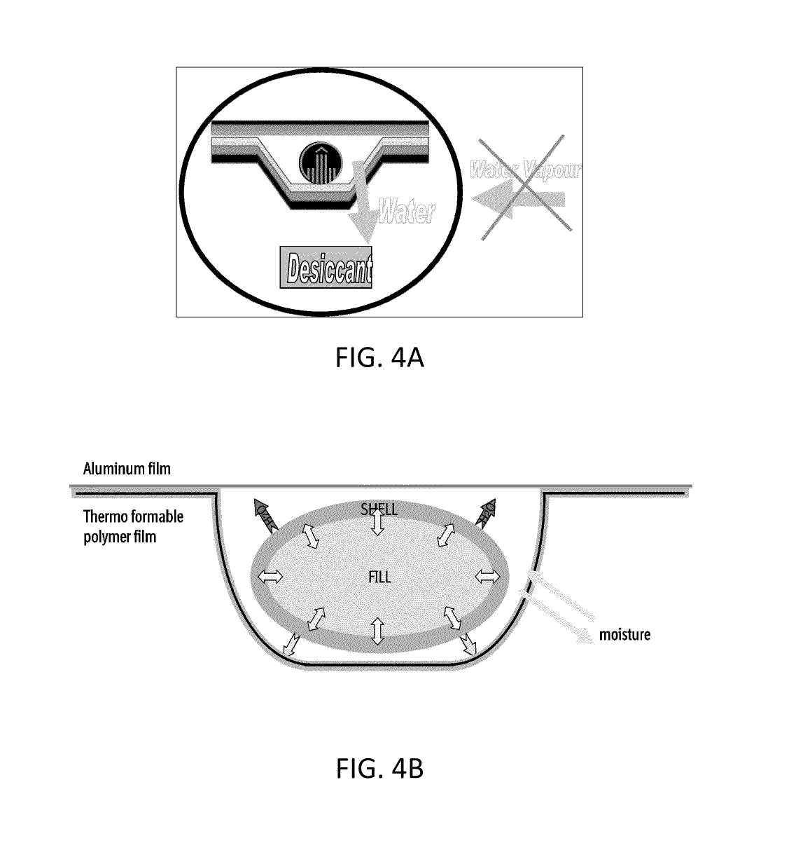

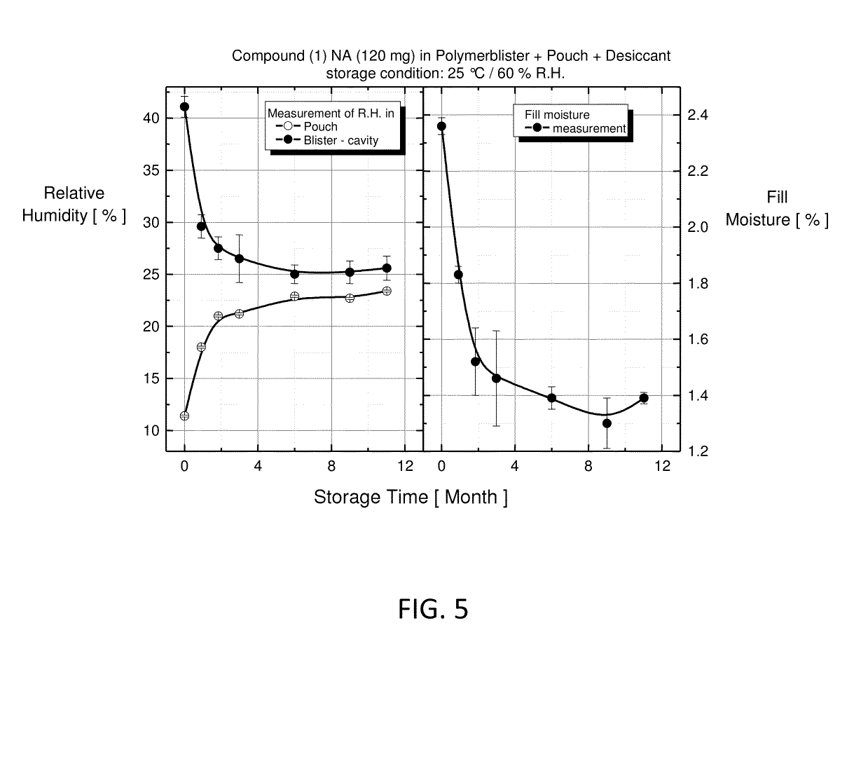

It is also preferred to use drug product packaging with high resistance to moisture ingress, such that the water content of Compound (1) NA capsules is maintained essentially constant during refrigerated storage. This allows the rate of Compound X formation to stay essentially constant and in balance with the rate of Compound X degradation, thereby maintaining the low steady state level of this impurity. One example of such packaging is a blister system incorporating desiccant material to further dry capsules during storage. The use of a pervious polymer blister enclosed in Alu foil with desiccant provides for an economic solution for moisture control and protection in a blister-packed product. FIG. 4A depicts a graphic representation of such blister system incorporating a desiccant in the product packaging and the affect on water transmission and FIG. 4B shows a more detailed depiction of the capsule in an exemplary polymer blister packaging system and the water transmission in such system. In one example, the use of a polymer blister system with desiccant quickly reduced the water content to approximately 1.5% in the capsule fill formulation and allowed the maintenance of this low water content during storage. See FIG. 5 depicting the changes in relative humidity over time within the pouch and within the capsule blister cavity and the changes in moisture content within the capsule fill formulation over the same storage period. In this example, the packaging used comprised an aluminum pouch containing a conditioned desiccant and Compound (1) NA capsules packed in a low moisture barrier thermo formable polymer film (e.g. poly vinyl chloride film) blister system)

Thus, in one general embodiment, the composition is stored in a moisture resistant packaging, optionally including a desiccant material. In addition to the polymer blister enclosed in Alu foil mentioned above, other commercially available moisture resistant packaging materials may be used, optionally along with conventional desiccant materials, for the purpose of maintaining reduced water content levels. Additional examples of packaging materials that may be used include thermo formable polychloride trifluoric ethylene (PCTFE) polymer films and alternative materials that show water vapor transmission rates below 0.1 g/m² d, including, e.g. ACLAR® 300 blisters and HPDE bottles.

One preferred embodiment of the invention combines the moisture control and temperature control methods to achieve degradation product control. For SEDDs capsules, the product should be dried during manufacture to less than about 3.0% water content, and once manufactured it is preferred to implement refrigerated storage for the bulk capsules (as outlined above) and to package them in moisture resistant packaging as early after manufacture as economically feasible. Doing so insures the lowest possible levels of Compound X in the drug product.

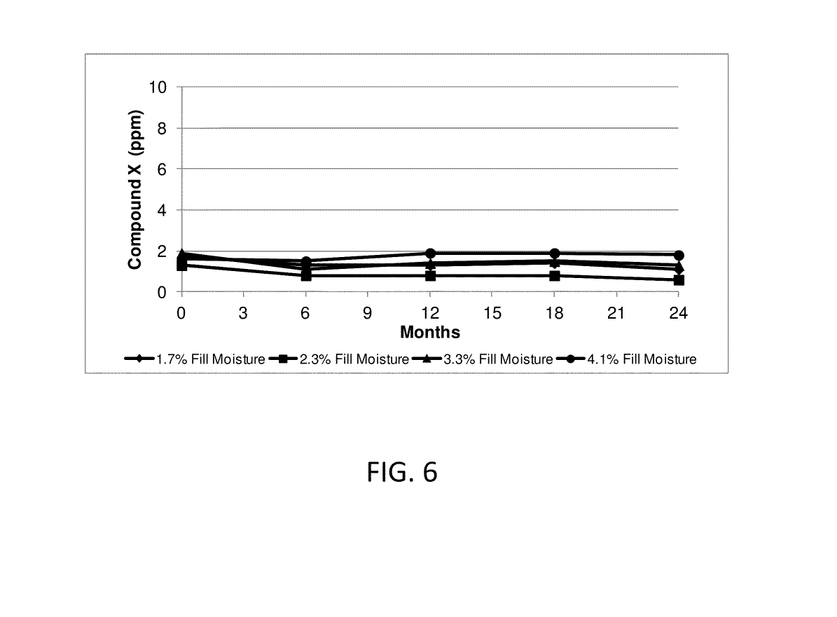

It has even been discovered that in formulations with even higher water content than 3%, the level of Compound X can be maintained below the limit from regulatory authorities by use of refrigeration in a moisture protective package. See FIG. 6 showing Compound X stability over a 24 month period under refrigerated conditions (4-5° C.) for 120 mg capsules of Compound (1) NA having different levels of fill water content, clearly demonstrating effective control even at higher water content levels under refrigerated conditions.

### 3. Excipient Control

It has been further discovered that Compound X is unstable and further degrades in the formulation and also reacts with fatty acid-based excipient materials, such as capmul and cremophor, to produce products that are not genotoxic as shown below:

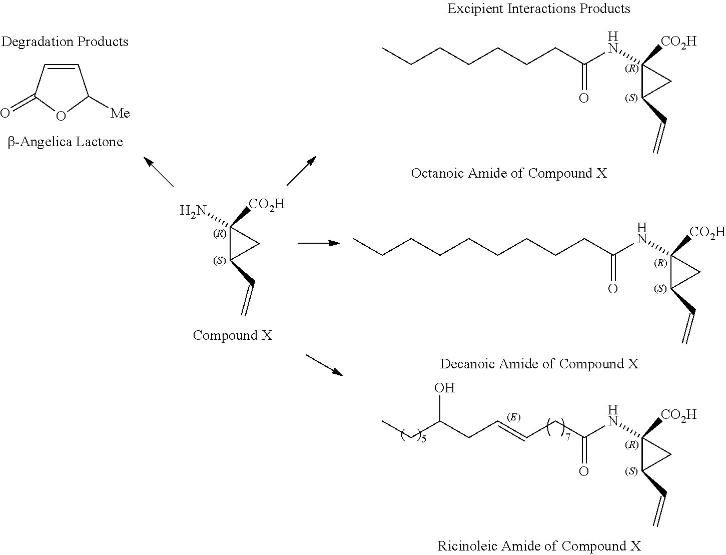

These subsequent degradation reactions result in the removal of Compound X and counter-balance the rate of Compound X formation. At refrigerated temperatures and under conditions of controlled moisture, the rates of formation and elimination of Compound X have been demonstrated to balance resulting in a steady state level of Compound X of approximately 0.5-1 ppm in drug product capsules. The above secondary reactions with fatty acid excipients has also been studied to be directly correlated with temperature. This provides an advantage under room temperature storage during patient use by maintaining Compound X levels well below allowable safety thresholds.

In view of the ability of fatty acids to contribute to the degradation of Compound X, the use of a fatty-acid based excipient material in the formulation constitutes an additional preferred embodiment of the present invention and an additional method for controlling the level of Compound X degradation product. Specific examples of fatty acids that may be used include capric acid, caprylic acid and ricinoleyl acid, although other fatty acids may also be suitable.

Experimental evidence for the reaction of Compound X with fatty acid-based excipient materials to form further degradation products as described above has also been obtained via spiking experiments.

### 4. Capsule Shell Control

It has been further discovered that for liquid formulations, e.g. SEDDS, contained within a soft gelatin capsule, the level of citric acid excipient contained within the gelatin capsule shell material has an affect on the level of Compound X formation within the fill formulation upon storage. This effect was demonstrated via a spiking experiment comparing Compound X formation in a liquid fill formulation stored in the presence of gelatin capsule shell material, the formulation being spiked with citric acid vs. without citric acid spiking. See the results in the table below (the capsule fill formulation used corresponds to that described in Example 1 of U.S. Patent Application Publication 2011/0160149):

| | Determined amount | |
|---|---|---|
| | of Compound X | |
| | | 1 Month storage |
| Sample Description | Time Zero | at 30° C. |
| Capsule shell material suspended in active | <1 ppm | <1 ppm |
| capsule fill formulation | | |
| Capsule shell material suspended in active | <1 ppm | 3 ppm |
| capsule fill formulation spiked with | | |
| approximately 1% citric acid | | |

As shown above, in the absence of citric acid there is stability upon 1 month storage of the formulation, whereas in the presence of only 1% citric acid there was an increase in the level of Compound X from less than 1 ppm to 3 ppm over the same storage period.

Although the specific mechanism by which citric acid contributes to Compound X formation is yet to be fully elucidated, a preferred embodiment is therefore to use a capsule shell that is substantially free of citric acid in order to minimize the formation of this degradation product. In this context, the term “substantially free” means less than about 1% of citric acid present in the capsule shell material.

### 5. Basification

Mechanistic studies have demonstrated that Compound X formation occurs via an acid catalyzed hydrolysis reaction. Formation of Compound X has not been observed under basic conditions. Modulation of formulation pH is therefore a potential means to reduce or eliminate formation of Compound X in liquid drug product formulations. The concept has been demonstrated in oral liquid formulations in which Tris has been included as a basifier.

Thus, an additional embodiment of the invention is a method for controlling the level of degradation product Compound X in a liquid pharmaceutical composition comprising a Compound (1), or a pharmaceutically acceptable salt thereof, and at least one pharmaceutically acceptable excipient, said method comprising adding a basifier to said composition to achieve an internal apparent pH of greater than about 7. In another embodiment, basifier is added to said composition to achieve an internal apparent pH of greater than about 8.

Basifiers that may be used include, for example, Tris (Tromethamine), Meglumine, Carbonate buffer and Arginine. Basifiers can be added to the formulation, dissolved in Water or in some of the co-solvents such as Polyethylene Glycol 400 or Propylene Glycol

The term “apparent pH” is in reference to the pH measurement obtained when using a standard pH electrode/meter to measure the pH of a non-aqueous solution, and is well understood in the art. See, e.g., USP Chapter <791>pH. Where a pH meter is standardized by use of an aqueous buffer and then used to measure the “pH” of a nonaqueous solution or suspension, the ionization constant of the acid or base, the dielectric constant of the medium, the liquid-junction potential (which may give rise to errors of approximately 1 pH unit), and the hydrogen-ion response of the glass electrode are all changed. For these reasons, the values so obtained with solutions that are only partially aqueous in character can be regarded only as apparent pH values. The term “apparent pH” is used herein, therefore, when referring to the pH value of a non-aqueous or only partially aqueous solution. An example of this type of formulation would be the oral solution formulation designed for pediatric use disclosed in WO 2010/059667. Thus, in a specific embodiment, the basifier is added to a water-containing solution designed for oral administration in order to achieve an internal apparent pH of greater than about 7. By using this technique, a level of degradation product in the oral solution is controlled.

To demonstrate this effect, the below table provides the storage stability results (level of Compound X) under various storage conditions for two oral solution formulations, one having no basifier added (F248) and the other having an added basifier (F383; containing Tris). The results demonstrate that the addition of a basifier results in a reduced level of degradation product being formed upon 12 months of storage.

| | | 6 m 40° C./ | 12 m 25° C./ | 12 m 30° C./ |
|---|---|---|---|---|
| Batches | Description | 75% RH | 60% RH | 75% RH |
| F 248 | Formula without | <2 ppm | 3.3 ppm | 3.3 ppm |
| | basifier | | | |
| F 383 | Formula with | 2.1 ppm | 0.9 ppm | 2.0 ppm |
| | basifier | | | |

Below are the formulas for the two tested oral solution formulations:

### Formula 248:

| INGREDIENT | g/100 g |
|---|---|
| Compound (1) Na | 4.4 |
| PEG 400 | 40.1 |
| Propylene Glycol | 5.7 |
| Vitamin E TPGS | 28.6 |
| Capmul MCM | 2.9 |
| Water | 14.4 |
| Sucralose | 1.9 |
| Butter mint | 1.0 |
| Butter toffee | 1.0 |
| Total | 100.0 |

### Formula 383:

| INGREDIENT | g/100 g |
|---|---|
| Compound (1) Na | 4.6 |
| PEG 400 | 54.6 |
| Propylene Glycol | 5.4 |
| Vitamin E TPGS | 13.4 |
| Water | 17.9 |
| Tris | 0.2 |
| Sucralose | 1.9 |
| Butter toffee | 2.0 |
| Total | 100 |

### 6. Reconstitution Approach

If the liquid pharmaceutical formulation of Compound (1) NA is to contain water as an excipient or co-solvent, a reconstitution approach is one additional method that has been to successfully used to control degradation product formation. As the rate of hydrolytic Compound X formation from Compound (1) NA is dependent on the presence of water to drive the reaction, ultra dry formulations can minimize Compound X formation. This has been demonstrated, for example, by the development of the Compound (1) NA oral solution drug product formulation that consists of two solutions designed to be mixed at time of patient use: Compound (1) NA dissolved in a dry solvent (e.g. PEG400 and propylene glycol) and an excipient vehicle containing water, the so-called “Reconstitution Approach”. By limiting or eliminating the association of Compound (1) NA with significant quantities of water during the storage period, the rate of Compound X formation during storage is greatly reduced.

In this method, a first non-aqueous concentrate formulation (the “first formulation pre-mixture”) is prepared comprising only Compound (1), or a pharmaceutically acceptable salt thereof, and non-aqueous based excipients, along with a second aqueous formulation pre-mixture (the “second formulation pre-mixture”) comprising water as an excipient. The first and second formulation pre-mixtures are then mixed to prepare the final formulation prior to patient use.

In one general embodiment, if the liquid pharmaceutical composition is to contain water as an excipient material, the level of degradation product X is controlled by first preparing a first formulation pre-mixture comprising only Compound (1), or pharmaceutically acceptable salt thereof, and non-aqueous based excipients and a second formulation pre-mixture comprising water as an excipient, and then mixing the first and second formulation pre-mixtures to prepare the final formulation prior to patient use.

To demonstrate this effect, the below table provides the storage stability results (level of Compound X) under various storage conditions for two oral solution formulations: one having no Compound X control method applied (F248) and the other being a concentrate to be reconstituted (F412) in which Compound (1) NA is dissolved in a non-aqueous-based system (=a “first formulation pre-mixture” described above). The results demonstrate that there is a greatly reduced level of degradation product formation in the concentrate as compared to the ready-to-use formulation having no control method applied.

| Batches | Description | 6 m 40/75 | 12 m 25/60 | 12 m 30/75 |
|---|---|---|---|---|
| F 248 | Ready to use solution | <2 ppm | 3.3 ppm | 3.3 ppm |
| | with no Compound | | | |
| | X control method | | | |
| F 412 | Concentrate to be | 0.1 ppm | ND | 0.1 ppm |
| | reconstituted | | | |

ND: non detectable

The formula for the F248 solution is as described above and below is the formula for the concentrate solution F412:

### Formula 412:

| INGREDIENT | g/100 g |
|---|---|
| Compound (1) Na | 14.9 |
| PEG 400 | 67.6 |
| Propylene Glycol | 17.5 |
| Total | 100 |

### Additional Embodiments of the Invention

### 1. Additional Method Embodiments

Additional embodiments of the present invention includes control methods incorporating to any combination of one or more of the above-described control methods as suitable for the particular composition at hand.

For example, in one preferred embodiment, when the liquid pharmaceutical composition is contained within a capsule one or both of the following methods are applied to the capsule to control the level of degradation product X: (a) it is dried such that it has a water content of less than about 3.0% w/w and stored under conditions sufficient to maintain a water content of less than about 3.0% w/w; and/or (b) it is stored in at a temperature of between about 2 and 8 degrees Celsius. In sub-embodiments, the water content is less than about 2.5%, or less than about 2.0%, and is stored under conditions sufficient to maintain such water content level. In additional preferred sub-embodiments, the composition contains a fatty acid excipient and/or the capsule shell material is substantially free of citric acid.

In another preferred embodiment, when the liquid pharmaceutical composition is a water-containing solution designed for oral administration, one of the following methods are applied:
(1) adding a basifier to said composition to achieve an internal apparent pH of greater than about 7, preferably greater than about 8;
(2) first preparing a first formulation pre-mixture comprising only Compound (1), or pharmaceutically acceptable salt thereof, and non-aqueous based excipients and a second formulation pre-mixture comprising water as an excipient, and then mixing the first and second formulation pre-mixtures to prepare the final formulation prior to patient use.

### 2. Additional Pharmaceutical Composition Embodiments

Additional embodiments are directed to a liquid pharmaceutical composition comprising a Compound (1), or a pharmaceutically acceptable salt thereof, and at least one pharmaceutically acceptable excipient, wherein the amount of degradation product Compound X in the composition is below a level of about 400 μg, or below a level of about 200 μg, or below a level of about 60 μg, or below a level of about 20 μg, when the composition contains a full daily dose of Compound (1) or pharmaceutically acceptable salt thereof in either single or multiple dosage units. In more specific embodiments, the amount of degradation product Compound X in the composition is below a level of about 10 μg or below a level of about 1.5 μg when the composition contains a full daily dose of Compound (1) or pharmaceutically acceptable salt thereof in either single or multiple dosage units.

Additional embodiments of the present invention are directed to the above-described liquid pharmaceutical compositions that may be prepared or treated using the above-described control methods, i.e., having low water content, low temperature, excipient controls and/or elevated pH.

Thus, additional embodiments of the present invention include liquid pharmaceutical compositions as described above comprising Compound (1), or a pharmaceutically acceptable salt thereof, and having the stated low levels of Compound X, wherein the composition has one or more of the following properties:
(a) a water content of less than about 3.0% w/w, or less than about 2.5% w/w, or less than about 2.0% w/w, optionally stored in moisture resistant packaging and optionally further including a desiccant material;
(b) an internal temperature of between about 2 and 8 degrees Celsius
(c) an internal apparent pH of greater than about 7, or greater than about 8;
(d) comprising a fatty acid excipient;
(e) contained within a capsule shell wherein the capsule shell material is substantially free of citric acid.

In additional more specific embodiments, the liquid pharmaceutical composition as described above comprising Compound (1), or a pharmaceutically acceptable salt thereof, and having the stated low levels of Compound X, has been stored for a period of at least 6 months, or at least 1 year, or at least 2 years, or at least 3 years, under conditions sufficient to maintain one or more of the following properties:
(a) a water content of less than about 3.0% w/w;
(b) an internal temperature of between about 2 and 8 degrees Celsius;
(c) a internal apparent pH of greater than about 7.

In another preferred embodiment, the pharmaceutical composition has (a) a water content of less than about 3.0% w/w, or less than about 2.5% w/w, or less than about 2.0% w/w and is stored in moisture resistant packaging optionally further including a desiccant material; and/or has (b) an internal temperature of between about 2 and 8 degrees Celsius. In a particular sub-embodiment, such composition is contained within a capsule. In another particular sub-embodiments thereof, the composition further comprises a fatty acid excipient and/or the capsule shell material is substantially free of citric acid.

In another preferred embodiment, the pharmaceutical composition has an internal apparent pH of greater than about 7, or greater than about 8. In a particular sub-embodiment, the liquid pharmaceutical composition is a water-containing solution designed for oral administration.

The degradation product control methods described herein can be used with various types of liquid formulations of Compound (1), including but not limited to the lipid-based SEDDS formulations described in US Publication 2011/0160149, and the oral solution formulations described in WO 2010/059667.

Additional embodiments are directed to the packaged dosage forms containing any of the aforementioned pharmaceutical compositions. Such embodiments include, for example, a liquid pharmaceutical composition in the form one or more discrete dosage units contained within a packaging, wherein the packaging further comprises written instructions for use to indicating that the composition should be stored at a temperature in the range of from 2 to 8 degrees Celsius. In a preferred sub-embodiment thereof, the packaged dosage units are stored together with a temperature monitoring device to measure and record the environmental temperature during storage or shipment.

### 3. SEDDS Formulation Embodiments

Embodiments of the SEDDS lipid-based formulations, e.g. as described in US Publication 2011/0160149, include:

Pharmaceutical compositions comprising Compound (1), or a pharmaceutically acceptable salt thereof, together with one or more pharmaceutically acceptable lipids and hydrophilic surfactants. The compositions may optionally include one or more additional ingredients, e.g., pharmaceutically acceptable hydrophilic solvents, solidifying agents, antioxidants, etc., as will be discussed in more detail below. The pharmaceutical compositions are liquid or semi-solid and are preferably encapsulated in a capsule for oral administration.

The composition may be characterized by one or more of the following features:
(1) either substantially free of any amine compound, or not containing any amine compound;
(2) either substantially free of any alcohol compound, or not containing any alcohol compound;
(3) either substantially free of any triglyceride compound, or not containing any triglyceride;
(4) either substantially free of any glyceride of a long chain fatty acid, or not containing any such glyceride;
(5) either substantially free of any additional surfactant compound, or not containing any additional surfactant compound;

A particular embodiment of this composition is directed to a pharmaceutical composition, comprising (or consisting essentially of):
(a) about 5% to 30% by weight of a compound of formula (1) or a pharmaceutically acceptable salt thereof;
(b) about 30% to 60% by weight of a pharmaceutically acceptable lipid;
(c) about 20% to 50% by weight of a pharmaceutically acceptable hydrophilic surfactant;
(d) optionally up to about 30% by weight of a pharmaceutically acceptable hydrophilic solvent;

A further particular embodiment of the composition is directed to a pharmaceutical composition, comprising (or consisting essentially of):
(a) about 10% to 20% by weight of a compound of formula (1) or a pharmaceutically acceptable salt thereof;
(b) about 40% to 50% by weight of a pharmaceutically acceptable lipid;
(c) about 25% to 35% by weight of a pharmaceutically acceptable hydrophilic surfactant;
(d) about 5% to 15% by weight of a pharmaceutically acceptable hydrophilic solvent;

A further particular embodiment of the composition is directed to a pharmaceutical composition, comprising (or consisting essentially of):
(a) about 5% to 30% by weight of a compound of formula (1) or a pharmaceutically acceptable salt thereof;
(b) about 30% to 60% by weight of a pharmaceutically acceptable lipid selected from fatty acids, medium or long chain mono-, di- or triglycerides, propylene glycol fatty acid esters, sorbitol fatty acid esters, water insoluble vitamins, and mixtures thereof;
(c) about 20% to 50% by weight of a pharmaceutically acceptable hydrophilic surfactant selected from polyethoxylated vegetable oils, polyethoxylated tocopherols, polyethoxylated sorbitol fatty acid esters, bile salts, lecithins and mixtures thereof;
(d) optionally up to about 30% by weight of a pharmaceutically acceptable hydrophilic solvent selected from propylene glycol, polypropylene glycol, polyethylene glycol, glycerol, ethanol, dimethyl isosorbide, glycofurol, propylene carbonate, dimethyl acetamide, water, or mixtures thereof;

A further particular embodiment of the composition is directed to a pharmaceutical composition, comprising (or consisting essentially of):
(a) about 10% to 20% by weight of a compound of formula (1) as the sodium salt;
(b) about 40% to 50% by weight of a pharmaceutically acceptable lipid selected from monoglycerides of caprylic and capric fatty acids; diglycerides of caprylic and capric fatty acids, and mixtures thereof;
(c) about 25% to 35% by weight of a pharmaceutically acceptable hydrophilic surfactant selected from tocopheryl polyethylene glycol succinate, polyoxyl 40 hydrogenated castor oil, and polyoxyl 35 castor oil and mixtures thereof;
(d) about 5% to 10% by weight of a pharmaceutically acceptable hydrophilic solvent selected from propylene glycol, polyethylene glycol, ethanol, water, and mixtures thereof.

### 4. Oral Solution Formulation Embodiments

Embodiments of the SEDDS lipid-based formulations, e.g. as described in WO 2010/059667, include:

A liquid composition comprising:

(a) Compound (1), or a pharmaceutically acceptable salt thereof:
(b) at least one surfactant; and
(c) at least one pharmaceutically acceptable solvent;
and wherein the composition is substantially free of lipid.

Additional embodiments of the composition may include:
(a) compositions wherein the weight ratio of surfactant to drug substance is greater than or equal to 1.4;
(b) compositions wherein the weight ratio of surfactant to drug substance is greater than or equal to 2.7; and
(c) compositions wherein the weight ratio of surfactant to drug substance is greater than or equal to 4.3.

Additional preferred embodiments under embodiments (a) to (c) above include:
(d) wherein under embodiment (b) above the compositions contain drug substance in an amount less than or equal to 4.6% and the weight ratio of surfactant to drug substance is greater than or equal to 2.7; and
(e) wherein under embodiment (c) above the compositions contain drug substance in an amount less than or equal 6.3% and the weight ratio of surfactant to drug substance is greater than or equal to 4.3.

In one preferred embodiment, the pharmaceutical composition comprises:

(a) 1% to 40% by weight of Compound (1), or a pharmaceutically acceptable salt thereof;
(b) 2% to 50% by weight of surfactant; and
(c) 10% to 90% by weight of solvent or mixture of solvents; and
wherein the composition is substantially free of lipid, or more preferably does not contain any lipid.

In another preferred embodiment, the pharmaceutical composition comprises:

(a) 2% to 10% by weight of Compound (1), or a pharmaceutically acceptable salt thereof;
(b) 10% to 30% by weight of surfactant; and
(c) 60% to 90% by weight of solvent or mixture of solvents; and
wherein the composition is substantially free of lipid, or more preferably does not contain any lipid.

In another preferred embodiment, the pharmaceutical composition comprises:

(a) 2% to 10% by weight of Compound (1), or a pharmaceutically acceptable salt thereof;
(b) 10% to 30% by weight of Vitamin E TPGS; and
(c) 60% to 90% by weight of a mixture of water, propylene glycol and polyethylene glycol 400; and
wherein the composition is substantially free of lipid, or more preferably does not contain any lipid.

In another preferred embodiment, the pharmaceutical composition comprises:

(a) 2% to 10% by weight of Compound (1), or a pharmaceutically acceptable salt thereof;
(b) 10% to 30% by weight of Vitamin E TPGS; and
(c) 60% to 90% by weight of a mixture of water and polyethylene glycol 400; and
wherein the composition is substantially free of lipid, or more preferably does not contain any lipid.

Additional embodiments include any of the above four embodiments, wherein the composition is (1) substantially free of propylene glycol or does not contain propylene glycol, and/or (2) substantially free of an amine or does not contain an amine.

### 4. Kit Embodiments

The invention also comprises a kit comprising two formulation pre-mixtures to be used in connection with the above-described Reconstitution Approach. The formulation pre-mixtures are packaged and sold together and the patient reconstitutes the final formulation by mixing together the two pre-mixtures prior to use. In a general embodiment, therefore, the kit comprises:
(a) a first formulation pre-mixture comprising a Compound (1), or a pharmaceutically acceptable salt thereof, and one or more non-aqueous based excipients; and;
(b) a second formulation pre-mixture comprising water as an excipient, and optionally one or more additional excipients.

Examples of final formulations that may be prepared using the reconstitution method include the oral solution formulations set forth above and those described in WO 2010/059667.

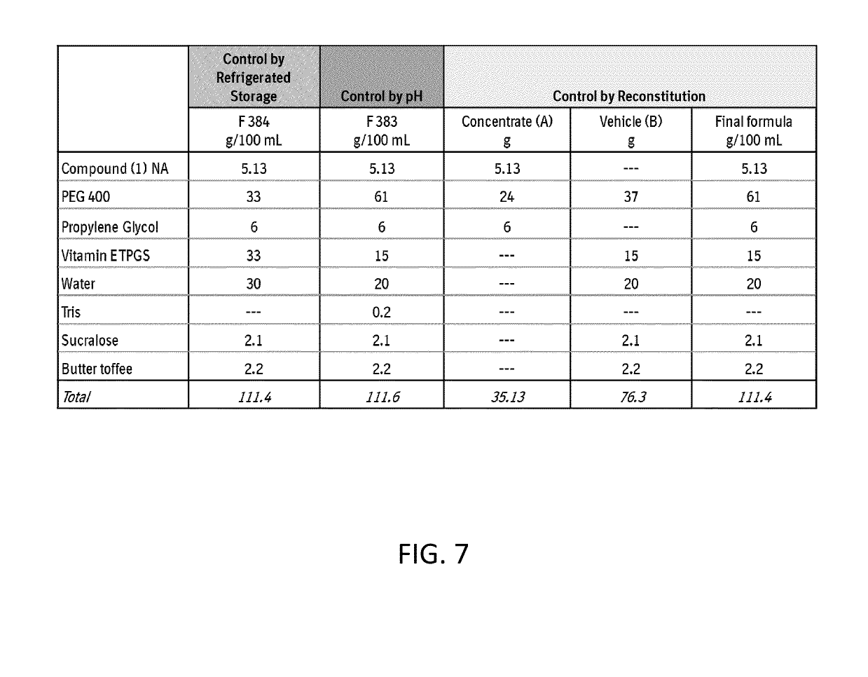

FIG. 7 sets forth three examples of different oral solution formulations designed for employing three different degradation control methods as described herein: wherein “Control by Refrigerated Storage”=temperature control method; “Control by pH”=basification control method; “Control by Reconstitution”=reconstitution control method.

### 5. Degradation Product Level Embodiments

The various techniques described herein may be employed, either separately or one or more of them together, to control the level of Compound X in the composition. In one embodiment of the invention the level of degradation product X is controlled to below a level of about 400 μg, or below a level of about 200 μg, or below a level of about 60 μg, or below a level of about 20 μg, when the composition contains a full daily dose of the active ingredient, and in another embodiment below a level of about 10 μg. Thus, specific embodiments of the present invention are directed to employing one or more of the methods described herein wherein the resulting amount of degradation product X in the composition is below a level of about 400 μg, or below a level of about 200 μg, or below a level of about 60 μg, or below a level of about 20 μg, or below a level of about 10 μg, when the composition contains a full daily dose of Compound (1), or pharmaceutically acceptable salt thereof, in either single or multiple dosage units.

As described above, the EMEA (European Medicines Agency) Guideline on the Limits of Genotoxic Impurities (28 Jun. 2006) specifies a maximum intake value of 1.5 μg/day of a genotoxic impurity as being associated with an acceptable risk for most pharmaceuticals. Accordingly, one additional preferred embodiment of the present invention is directed to employing one or more of the methods described herein wherein the resulting amount of degradation product X in the composition is below a level of about 1.5 μg when the composition contains a full daily dose of Compound (1), or pharmaceutically acceptable salt thereof, in either single or multiple dosage units.

For example, when a full daily dose is 240 mg of Compound (1), this intake value (1.5 μg/day) calculates to a level of 6 ppm (parts-per-million). Thus, an additional embodiment is wherein the resulting amount of degradation product X in the composition is below a level of about 6 ppm for each 240 mg of Compound (1) or pharmaceutically acceptable salt thereof. Preferred subembodiments at such dosage include upper limits of 3 ppm, or 2 ppm or 1 ppm.

As an additional example, when a full daily dose is 120 mg of Compound (1), this intake value (1.5 μg/day) calculates to a level of 12 ppm (parts-per-million). Thus, an additional embodiment is wherein the resulting amount of degradation product X in the composition is below a level of about 12 ppm for each 120 mg of Compound (1) or pharmaceutically acceptable salt thereof. Preferred subembodiments at such dosage include upper limits of 8 ppm, or 4 ppm or 2 ppm.

1. A liquid pharmaceutical composition comprising a compound of formula (1): 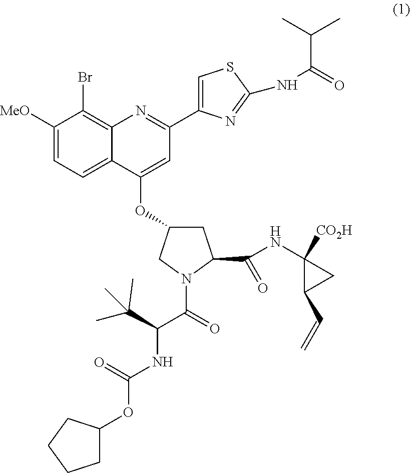 [SMILES: C=C[C@@H]1C[C@]1(NC(=O)[C@@H]1C[C@@H](Oc2cc(-c3csc(NC(=O)C(C)C)n3)nc3c(Br)c(OC)ccc23)CN1C(=O)[C@@H](NC(=O)OC1CCCC1)C(C)(C)C)C(=O)O]
or a pharmaceutically acceptable salt thereof, and at least one pharmaceutically acceptable excipient, wherein the amount of degradation product Compound X: 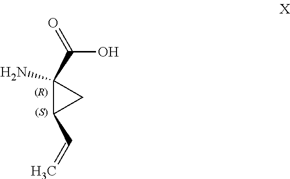
in the composition is below a level of about 400 μg when the composition contains a full daily dose of Compound (1) or pharmaceutically acceptable salt thereof in either single or multiple dosage units.

2. A liquid pharmaceutical composition according to claim 1, wherein the composition has one or more of the following properties:
(a) a water content of less than about 3.0% w/w;
(b) an internal temperature of between about 2 and 8 degrees Celsius;
(c) a internal apparent pH of greater than about 7.

3. A liquid pharmaceutical composition according to claim 2, wherein the composition has been stored for a period of at least 6 months under conditions sufficient to maintain one or more of the following properties:
(a) a water content of less than about 3.0% w/w;
(b) an internal temperature of between about 2 and 8 degrees Celsius;
(c) a internal apparent pH of greater than about 7.

4. A liquid pharmaceutical composition according to claim 2, wherein the composition has a water content of less than about 3.0% w/w.

5. A liquid pharmaceutical composition according to claim 4, wherein the composition is stored in moisture resistant packaging optionally further including a desiccant material.

6. A liquid pharmaceutical composition according to claim 2, wherein the composition has an internal temperature of between about 2 and 8 degrees Celsius

7. A liquid pharmaceutical composition according to claim 2, wherein the liquid pharmaceutical composition is contained within a capsule and has one or both of the following properties: (a) a water content of less than about 3.0% w/w; and (b) an internal temperature of between about 2 and 8 degrees Celsius.

8. A liquid pharmaceutical composition according to claim 7, wherein the capsule shell material is substantially free of citric acid

9. A liquid pharmaceutical composition according to claim 1, wherein the composition contains a fatty acid excipient

10. A liquid pharmaceutical composition according to claim 2, wherein the composition has an internal apparent pH of greater than about 7.

11. A liquid pharmaceutical composition according to claim 10, wherein the composition is a water-containing solution designed for oral administration and wherein the composition is optionally stored at a temperature of between about 2 and 8 degrees Celsius.

12. A liquid pharmaceutical composition according to claim 1, wherein the composition is in the form one or more discrete dosage units contained within a packaging, wherein the packaging further comprises written instructions for use indicating that the composition should be stored at a temperature in the range of from 2 to 8 degrees Celsius

13. A liquid pharmaceutical composition according to claim 12, wherein the packaged dosage units are stored together with a temperature monitoring device to measure and record the environmental temperature during storage or shipment.

14. A method for controlling the level of degradation product Compound X: 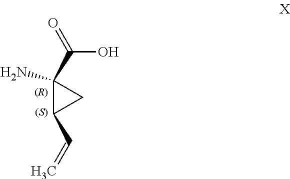
in a liquid pharmaceutical composition comprising a compound of formula (1): 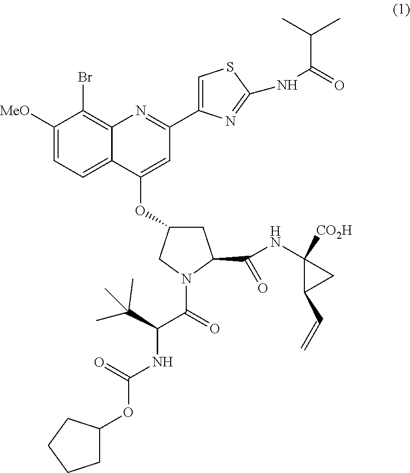 [SMILES: C=C[C@@H]1C[C@]1(NC(=O)[C@@H]1C[C@@H](Oc2cc(-c3csc(NC(=O)C(C)C)n3)nc3c(Br)c(OC)ccc23)CN1C(=O)[C@@H](NC(=O)OC1CCCC1)C(C)(C)C)C(=O)O]
or a pharmaceutically acceptable salt thereof, and at least one pharmaceutically acceptable excipient, said method comprising one or more of the following:
(a) drying said composition such that it has a water content of less than about 3.0% w/w and storing the composition under conditions sufficient to maintain a water content of less than about 3.0% w/w;
(b) storing said composition at a temperature of between about 2 and 8 degrees Celsius;
(c) adding a basifier to said composition to achieve an internal apparent pH of greater than about 7; or
(d) if the liquid pharmaceutical composition is to contain water as an excipient material, first preparing a first formulation pre-mixture comprising only Compound (1), or pharmaceutically acceptable salt thereof, and non-aqueous based excipients and a second formulation pre-mixture comprising water as an excipient, and then mixing the first and second formulation pre-mixtures to prepare the final composition just prior to patient use.

15. A method according to claim 14, wherein the resulting amount of degradation product Compound X in the composition is below a level of about 400 μg when the composition contains a full daily dose of Compound (1) or pharmaceutically acceptable salt thereof in either single or multiple dosage units.

16. A method according to claim 15, wherein the composition, or in the case of (d) the first-formulation pre-mixture, has been stored for a period of at least 6 months under conditions sufficient to maintain one or more of the following properties:
(a) a water content of less than about 3.0% w/w;
(b) an internal temperature of between about 2 and 8 degrees Celsius;
(c) a internal apparent pH of greater than about 7.

17. A method according to claim 14, wherein said composition is dried such that it has a water content of less than about 3.0% w/w and stored under conditions sufficient to maintain a water content of less than about 3.0% w/w.

18. A method according to claim 17, wherein the composition is stored in moisture resistant packaging, optionally further including a desiccant material.

19. A method according to claim 14, wherein said composition is stored at a temperature of between about 2 and 8 degrees Celsius.

20. A method according to claim 14, wherein the liquid pharmaceutical composition is contained within a capsule and one or both of the following methods are applied to the capsule: (a) it is dried such that it has a water content of less than about 3.0% w/w and stored under conditions sufficient to maintain a water content of less than about 3.0% w/w; and/or (b) it is stored in at a temperature of between about 2 and 8 degrees Celsius.

21. A method according to claim 20, wherein the capsule shell material is substantially free of citric acid

22. A method according to claim 14, wherein the composition contains a fatty acid excipient.

23. A method according to claim 14 comprising adding a basifier to said composition to achieve an internal apparent pH of greater than about 7.

24. A method according to claim 23, wherein the composition is a water-containing solution designed for oral administration and wherein the composition is optionally stored at a temperature of between about 2 and 8 degrees Celsius.

25. A kit comprising:
(a) a first formulation pre-mixture comprising a compound of formula (1): 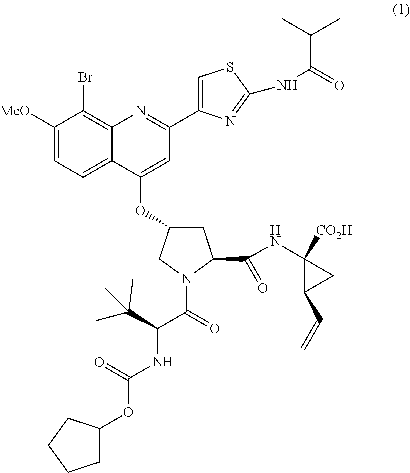 [SMILES: C=C[C@@H]1C[C@]1(NC(=O)[C@@H]1C[C@@H](Oc2cc(-c3csc(NC(=O)C(C)C)n3)nc3c(Br)c(OC)ccc23)CN1C(=O)[C@@H](NC(=O)OC1CCCC1)C(C)(C)C)C(=O)O]
or a pharmaceutically acceptable salt thereof, and one or more non-aqueous based excipients; and
(b) a second formulation pre-mixture comprising water as an excipient, and optionally one or more additional excipients.
